(19) United States
(12) Patent Application Publication
(10) Pub. No.: US 2013/0177959 A1
(43) Pub. Date: Jul. 11, 2013

(54) NOVEL CBH1-EG1 FUSION PROTEINS AND USE THEREOF

(71) Applicants: Senta Blanquet, Versailles (FR); Gaelle Brien, Montrouge (FR); Nicolas Lopes Ferreira, Montrouge (FR); Edward A. Bayer, Hashavim (IL); Sarah Morais, Ashdod (IL); Yoav Barak, Rehovot (IL); Jari Vehmaanpera, Klaukkala (FI); Taija Leinonen, Riihimaki (FI)

(72) Inventors: Senta Blanquet, Versailles (FR); Gaelle Brien, Montrouge (FR); Nicolas Lopes Ferreira, Montrouge (FR); Edward A. Bayer, Hashavim (IL); Sarah Morais, Ashdod (IL); Yoav Barak, Rehovot (IL); Jari Vehmaanpera, Klaukkala (FI); Taija Leinonen, Riihimaki (FI)

(73) Assignees: IFP ENERGIES NOUVELLES, RUEIL-MALMAISON CEDEX (FR); ROAL OY, RAJAMAKI (FI); YEDA RESEARCH AND DEVELOPMENT CO.LTD, REHOVOT (IL)

(21) Appl. No.: 13/634,924

(22) PCT Filed: Mar. 25, 2011
(86) PCT No.: PCT/IB2011/000927

§ 371 Date: Mar. 25, 2013

(30) Foreign Application Priority Data
Mar. 26, 2010 (FR) 10/52249

(51) Int. Cl. C12N 9/96; C12P 7/14

(52) U.S. Cl. CPC C12N 9/96; C12P 7/14
USPC 435/162; 536/23.2; 435/188; 435/320.1; 435/254.11; 435/254.3; 435/254.4; 435/254.6

(57) ABSTRACT

The object of the present invention are novel fusion proteins comprising enzymes degrading plant cell walls, and the use thereof in a method of producing ethanol from lignocellulosic biomass.

### FIELD OF THE INVENTION

The present invention relates to novel fusion proteins comprising enzymes that degrade plant cell walls, and to the use thereof in a method of producing ethanol from lignocellulosic biomass.

### BACKGROUND OF THE INVENTION

Lignocellulosic biomass represents one of the most abundant renewable resources on earth, and certainly one of the least expensive. The substrates considered are very varied since they concern both lignous substrates (broadleaved trees and coniferous trees), agricultural sub-products (straw) or sub-products from industries generating lignocellulosic waste (food-processing industries, paper industries).

Lignocellulosic biomass consists of three main polymers: cellulose (35 to 50%), hemicellulose (20 to 30%), which is a polysaccharide essentially consisting of pentoses and hexoses, and lignin (15 to 25%), which is a polymer of complex structure and high molecular weight, consisting of aromatic alcohols linked by ether bonds.

These various molecules are responsible for the intrinsic properties of the plant wall and they organize into a complex entanglement.

The cellulose and possibly the hemicelluloses are the targets of enzymatic hydrolysis, but they are not directly accessible to enzymes. These substrates therefore have to undergo a pretreatment prior to the enzymatic hydrolysis stage. The pretreatment aims to modify the physical and physico-chemical properties of the lignocellulosic material in order to improve the accessibility of the cellulose stuck in the lignin and hemicellulose matrix. It can also release the sugars contained in the hemicelluloses as monomers, essentially pentoses, such as xylose and arabinose, and hexoses, such as galactose, mannose and glucose.

Ideally, the pretreatment must be fast and efficient, with high substrate concentrations, and material losses should be minimal. There are many technologies available: acidic boiling, alkaline boiling, steam explosion (Pourquié J. and Vandecasteele J. P. (1993) Conversion de la biomasse lignocellulosique par hydrolyse enzymatique et fermentation. Biotechnologie, 4^{th} ed., René Scriban, coordinateur Lavoisier TEC & DOC, Paris, 677-700), Organosolv processes, or twin-screw technologies combining thermal, mechanical and chemical actions (Ogier J. C. et al. (1999) Production d'éthanol à partir de biomasse lignocellulosique, Oil & Gas Science & Technology (54):67-94). The pretreatment efficiency is measured by the hydrolysis susceptibility of the cellulosic residue and by the hemicellulose recovery rate. From an economic point of view, the pretreatment preferably leads to total hydrolysis of the hemicelluloses, so as to recover the pentoses and possibly to upgrade them separately from the cellulosic fraction. Acidic pretreatments under mild conditions and steam explosion are well suited techniques. They allow significant recovery of the sugars obtained from the hemicelluloses and good accessibility of the cellulose to hydrolysis.

The cellulosic residue obtained is hydrolyzed via the enzymatic process using cellulolytic and/or hemicellulolytic enzymes. Microorganisms such as fungi belonging to the Trichoderma, Aspergillus, Penicillium, Schizophyllum, Chaetomium, Magnaporthe, Podospora, Neurospora genera, or anaerobic bacteria belonging for example to the Clostridium genus, produce these enzymes containing notably cellulases and hemicellulases, suited for total hydrolysis of the cellulose and of the hemicelluloses.

Enzymatic hydrolysis is carried out under mild conditions (temperature of the order of 45-50° C. and pH value 4.8) and it is efficient. On the other hand, as regards the process, the cost of enzymes is still very high. Considerable work has therefore been conducted in order to reduce this cost: i) first, increase in the production of enzymes by selecting hyperproductive strains and by improving fermentation methods, ii) decrease in the amount of enzymes in hydrolysis, by optimizing the pretreatment stage or by improving the specific activity of these enzymes. During the last decade, the main work consisted in trying to understand the mechanisms of action of the cellulases and of expression of the enzymes so as to cause secretion of the enzymatic complex which is best suited for hydrolysis of the lignocellulosic substrates by modifying the strains with molecular biology tools.

Filamentous fungi, as cellulolytic organisms, are of great interest to industrialists because they have the capacity to produce extracellular enzymes in very large amounts. The most commonly used microorganism for cellulase production is the Trichoderma reesei fungus. This fungus has the ability to produce, in the presence of an inducing substrate, cellulose for example, a secretome (all the proteins secreted) suited for cellulose hydrolysis. The enzymes of the enzymatic complex comprise three major types of activities: endoglucanases, exoglucanases and β-glucosidases.

Other proteins with essential properties for the hydrolysis of lignocellulosic materials are also produced by Trichoderma reesei, xylanases for example. The presence of an inducing substrate is essential for the expression of cellulolytic and/or hemicellulolytic enzymes. The nature of the carbon substrate has a strong influence on the composition of the enzymatic complex. This is the case of xylose which allows, associated with a cellulase inducing carbon substrate such as cellulose or lactose, a significant increase in the activity referred to as xylanase activity to be significantly improved.

Conventional genetic engineering techniques using mutagenesis have allowed cellulase-hyperproductive Trichoderma reesei strains such as MCG77 (Gallo—U.S. Pat. No. 4,275 167), MCG 80 (Allen, A. L. and Andreotti, R. E., Biotechnol-Bioengi 1982, (12): 451-459), RUT C30 (Montenecourt, B. S. and Eveleigh, D. E., Appl. Environ. Microbiol. 1977, (34): 777-782) and CL847 (Durand et al., 1984, Proc. Colloque SFM “Génétique des microorganismes industriels”. Paris. H. HESLOT Ed, pp 39-50) to be selected. The improvements have allowed to obtain hyperproductive strains that are less sensitive to catabolic repression on monomer sugars notably, glucose for example, than wild type strains.

The fact that genetic engineering techniques intended to express heterologous genes within these fungal strains are now widely practised also opened up the way for the use of such microorganisms as hosts for industrial production.

New enzymatic profiling techniques made it possible to create very efficient host fungal strains for the production of recombinant enzymes on the industrial scale [Nevalainen H. and Teo V. J. S. (2003) Enzyme production in industrial fungi-molecular genetic strategies for integrated strain improvement. In Applied Mycology and Biotechnology (Vol. 3) Fungal Genomics (Arora D. K. and Kchachatourians G. G. eds.), pp. 241-259, Elsevier Science].

One example of this type of modification is the production of cellulases from a T. reesei strain [Harkki A. et al. (1991) Genetic engineering of Trichoderma to produce strains with novel cellulase profiles. Enzyme Microb. Technol. (13): 227-233; Karhunen T. et al. (1993) High-frequency one-step gene replacement in Trichoderma reesei. I. Endoglucanase I overproduction. Mol. Gen. Genet. 241, 515-522].

Another example is the production of fusion proteins between two enzymes playing complementary roles for the degradation of plant cell walls. Document WO-07/115,723 notably describes a fusion protein between a swollenin exhibiting no hydrolytic activity (but capable of breaking the hydrogen bonds between the cellulose chains or the cellulose microfibrills and other polymers of the plant wall) and a second enzyme exhibiting a hydrolytic activity. On the other hand, exo-endocellulasic heterologous fusion proteins also have to be mentioned within the scope of the present invention. Document WO-97/27,306 describes a fusion protein between a fungal CBH1 exo-cellobiohydrolase (this exo-cellobiohydrolase comprises its signal peptide and its catalytic region) and a E1, E2, E4 or E5 endoglucanase from the Thermobidifa fusca bacterium, said fusion protein being furthermore CBM-free. Similarly, document WO-07/019,949 describes exo-endocellulasic fusion proteins one of which contains a fungal CBH1 exo-cellobiohydrolase (wherein the signal peptide is that of feruloyl esterase A from Aspergillus niger), associated with another cell wall degrading enzyme, and possibly with a CBM. Finally, document EP-1,740,700 describes exo-endocellulasic fusion proteins that can contain the catalytic domain of an exo-cellobiohydrolase such as CBH1, an endoglucanase of nomenclature EC 3.2.1.4, possibly a CBM and a linker peptide. However, this application only specifically describes endonucleases from the Acidothermus cellulolyticus bacterium.

The present invention results from the discovery made by the inventors that their fusion proteins can, when mixed in particular proportions with a complete Trichoderma reesei enzymatic cocktail, degrade celllulosic and/or lignocellulosic substrates more efficiently than said enzymatic cocktail alone or than said fusion proteins of the present invention alone, in particular when the rate of dry matter of said cellulosic or lignocellulosic substrates is high. This result is particularly interesting within the context of processes such as bioethanol production from cellulosic and/or lignocellulosic substrates, and other processes wherein the amount of water required for the functioning of glycoside hydrolases such as cellobiohydrolases and endoglucanases is reduced.

### DETAILED DESCRIPTION

The object of the present invention thus are fusion proteins that degrade plant cell walls, said proteins comprising:
i) an enzyme that is a recombinant protein consisting of the catalytic domain of the exo-cellobiohydrolase CBH1, said enzyme having the sequence SEQ ID NO: 4, or functional fragment thereof, or of a functional mutated form thereof,
ii) an enzyme that is a recombinant protein consisting of the catalytic domain of the endoglucanase EG1, said enzyme having the sequence SEQ ID NO: 12, or functional fragment thereof, or of a functional mutated form thereof,
iii) a signal peptide, placed at the N-terminal end of said fusion protein upstream from the two enzymes mentioned in i) and ii), said signal peptide originating from fungal native cellulase or hemicellulase, or from native fungal cellulase belonging to the GH6 or GH7 family,
iv) a polysaccharide binding module originating from fungal native cellulase or hemicellulase, or from native fungal cellulase belonging to the GH6 or GH7 family and
   each constituent i), ii) and iv) is linked to one or two of the other constituents i), ii) and iv) at most, by at least one linker peptide of identical or different sequences made up of 10 to 100 amino acids.

What is referred to as “cellulase” is an enzyme such as an endoglucanase, an exoglucanase, a cellobiohydrolase or a β-glucosidase.

What is referred to as “hemicellulase” is an enzyme hydrolyzing the carbohydrates that make up the hemicelluloses, such as a xylanase.

What is referred to as “functional fragment” is a protein or a peptidic sequence obtained after truncation of the original protein or peptidic sequence, and which has a catalytic activity substantially identical to the catalytic activity of said entire protein or said original peptidic sequence. The term “functional fragment” comprises the “fragments” and “segments” of said entire protein or of said original peptidic sequence. In the definition of the functional fragment, the terms “protein” and “peptidic sequence” designate a contiguous chain of amino acids linked to each other by peptidic bonds.

What is referred to as “functional mutated form” is a protein or a peptidic sequence obtained after modifying the original protein or peptidic sequence, and which has a catalytic activity substantially identical to the catalytic activity of said entire protein or of said original peptidic sequence from which it originates. Said functional mutated form of the entire protein or of the original peptidic sequence may or not contain post-translational modifications such as a glycosylation if such a modification does not prevent the aforementioned biological activity. In the definition of the mutated functional form, the terms “protein” and “peptidic sequence” designate any contiguous chain containing several amino acids, linked to each other by peptidic bonds. The term “peptidic sequence” used in this definition also designates the short chains, commonly called peptides, oligopeptides and oligomers. Said functional mutated form may or not contain amino acids other than the 20 coded amino-acids such as, for example, hydroxyprolin or selenomethionin, as well as any other non-essential and non-proteinogen amino acid. Said functional mutated forms comprise those modified by natural processes, such as molecular maturation and the other post-translational modifications, and by chemical modification techniques. Such modifications are well described in the literature and known to the person skilled in the art. In the definition of the functional mutated form, the same type of modification can be present in the same protein or in the same peptidic sequence on several sites of said protein or of said peptidic sequence, and in various proportions. Besides, said protein or peptidic sequence can contain different types of modification.

What is referred to as “catalytic domain of a cellulase” is the module of the polypeptidic chain responsible for the hydrolytic action on the cellulosic or lignocellulosic substrate.

What is referred to as “GH6 or GH7 family” are the families of Glycoside Hydrolases (GH) No. 6 and 7 from the CAZY (Carbohydrate Active enZYme database) database classification. The CAZY base is accessible online (http://www.cazy.org/).

What is referred to as “signal peptide” is the fragment of the protein or of the peptide sequence of the cellulase or the hemicellulase it originates from, whose function is to direct the transport of said fusion protein to the extracellular medium of the host from which the protein originates, notably SEQ ID NO: 2 encoded by SEQ ID NO: 1.

What is referred to as “polysaccharide binding module” (CBM, Carbohydrate Binding Module) is a peptidic sequence having a sufficient affinity with the cellulose or the lignocellulose to anchor the native protein from which it originates on said cellulose. There are CBMs of type I, II or III, which are molecules well known to the person skilled in the art. The CBMs used in the present invention are preferably of type I, notably the peptidic sequence SEQ ID NO: 8 encoded by SEQ ID NO: 7, corresponding to the CBM of the exo-cellobiohydrolase CBH1.

What is referred to as “linker peptide” is a contiguous chain of 10 to 100 amino acids, preferably 10 to 60 amino acids. Linker peptides can optionally be used to link the various constituents of the fusion proteins mentioned from i) to iv) to each other. Thus, the signal peptide mentioned in iii) can only be linked to one constituent selected among i), ii) and iv), and each one of constituents i), ii) and iv) can only be linked to one or two other constituents i), ii) and iv) at most, by at least one linker peptide of identical or different sequences consisting of 10 to 100 amino acids.

In an advantageous embodiment of the invention, the functional mutated form of enzyme ii) has a sequence exhibiting at least 75%, advantageously at least 80% homology or identity, more advantageously at least 85% homology or identity, more advantageously yet at least 90% homology or identity, or 95% or 99% homology or identity with the sequence of the catalytic domain of said enzyme. All the forms exhibiting the aforementioned homologies or identities keep a catalytic activity substantially identical to the catalytic activity of the protein or of the original peptidic sequence from which they originate.

In a preferred embodiment, the linker peptides are selected from among the sequences of SEQ ID NOS: 6 and 10, respectively encoded by SEQ ID NOS: 5 and 9, and corresponding to the linker peptides of the exo-cellobiohydrolases CBH1 and CBH2 respectively.

Finally, in another embodiment, the linker peptides used are hyperglycosylated.

The fusion proteins are fusion proteins wherein the catalytic domain of the endoglucanase mentioned in ii) has the sequence SEQ ID NO: 12 encoded by SEQ ID NO: 11, corresponding to the catalytic domain of the Endoglucanase EG1 (EG1^{cat}) of T. reesei.

According to the invention, the enzyme mentioned in i) is processive; the enzyme mentioned in ii) is non processive.

What is referred to as “processive” is a cellulase that can achieve several cleavages in the cellulose or in the lignocellulose prior to detaching therefrom. A “non-processive” enzyme is defined within the scope of the present invention as an enzyme that randomly intersects within the non-crystalline regions of the cellulose polymer.

The fusion proteins are proteins wherein the enzyme mentioned in i) has the sequence SEQ ID NO: 4 encoded by SEQ ID NO: 3, corresponding to the catalytic domain of the exo-cellobiohydrolase CBH1 of T. reesei.

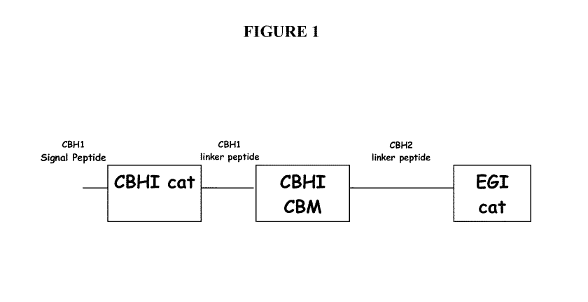

In another embodiment of the invention, the fusion protein has the complete sequence SEQ ID NO: 14 encoded by SEQ ID NO: 13, or a functional mutated form thereof. This sequence corresponds to the protein shown in FIG. 1, which is the fusion protein called “CBH1-EG1^{cat}”.

Another object of the present invention is a mixture for degrading the plant cell walls, which comprises a fusion protein according to any of the above definitions and a T. reesei enzymatic cocktail. What is referred to as “T. reesei enzymatic cocktail” is the secretome of T. reesei or a commercial mixture such as Econase®. This combination has been shown particularly advantageous for the degradation of substrates with a high dry matter content, as illustrated in Example 3.

In an advantageous embodiment of the invention, the fusion protein represents between 1 and 50 wt. % of the combination, more advantageously between 10 and 50%.

Isolated nucleic acids coding for a fusion protein according to any of the above definitions are another object of the invention, notably SEQ ID NO: 13.

Similarly, an expression vector comprising the nucleic acid molecule according to the above definition is also an object of the invention.

Another object of the present invention is a host cell containing the expression vector according to the above definition, said host cell being a cell of a fungus belonging to:
the ascomycetes, including the Aspergillus, Chaetomium, Magnaporthe, Podospora, Neurospora and Trichoderma genera, or
the basidiomycetes, including the Halocyphina, Phanerochaete and Pycnoporus genera.

In an even more advantageous embodiment, the host cell is a cell of a fungus selected from among the group consisting of: Aspergillus fumigatus, Aspergillus niger, Aspergillus tubingensis, Chaetomium globosum, Halocyphina villosa, Magnaporthe grisea, Phanerochaete chrysosporium, Pycnoporus cinnabarinus, Pycnoporus sanguineus, Trichoderma reesei.

Another object of the present invention is a method of preparing a fusion protein according to any one of the previous definitions, comprising:
in vitro cultivation of the host cell according to the above definition, and
recovery, optionally followed by purification of the fusion protein produced by said host cell.

Another object of the present invention is also the use of the novel fusion proteins according to any of the above definitions in an ethanol production process from cellulosic and lignocellulosic biomass.

The invention thus relates to an ethanol production method from cellulosic or lignocellulosic materials, comprising:
a) at least one cellulosic or lignocellulosic substrate pretreatment stage,
b) at least one stage of enzymatic hydrolysis of the pretreated substrate, then at least one stage of alcoholic fermentation of the hydrolysate obtained, wherein the enzymatic hydrolysis is carried out by the mixture of an enzymatic cocktail of a fungus secreted by a Trichoderma reesei strain and of a fusion protein consisting of two enzymes degrading the plant cell walls, said fusion protein representing between 1 and 50 wt. %, advantageously between 10 and 50 wt. % of said enzymatic cocktail and comprising:
   i) an enzyme that is a recombinant protein consisting of the catalytic domain of the exo-cellobiohydrolase CBH1, said enzyme having the sequence SEQ ID NO: 4, or functional fragment thereof, or of a functional mutated form thereof,
   ii) an enzyme that is a recombinant protein consisting of the catalytic domain of the endoglucanase EG1, said enzyme having the sequence SEQ ID NO: 12, or functional fragment thereof, or of a functional mutated form thereof,
   iii) a signal peptide, placed at the N-terminal end of said fusion protein upstream from the two enzymes mentioned in i) and ii), said signal peptide originating from fungal native cellulase or hemicellulase, or from native fungal cellulase belonging to the GH6 or GH7 family,
   iv) a polysaccharide binding module originating from fungal native cellulase or hemicellulase, or from native fungal cellulase belonging to the GH6 or GH7 family and
      each constituent i), ii) and iv) is linked to one or two of the other constituents i), ii) and iv) at most, by at least one linker peptide of identical or different sequences made up of 10 to 100 amino acids.

In another embodiment of the invention, the ethanol production method from cellulosic or lignocellulosic materials comprises:
a) at least one cellulosic or lignocellulosic substrate pretreatment stage,
b) at least one stage of enzymatic hydrolysis of the pretreated substrate, then at least one stage of alcoholic fermentation of the hydrolysate obtained, wherein the enzymatic hydrolysis is carried out by the mixture of an enzymatic cocktail of a fungus secreted by a Trichoderma reesei strain and of a fusion protein consisting of two enzymes degrading the plant cell walls, said fusion protein representing between 1 and 50 wt. %, advantageously between 10 and 50 wt. % of said enzymatic cocktail and comprising:
   i) an enzyme that is a recombinant protein consisting of the catalytic domain of the exo-cellobiohydrolase CBH1 of T. reesei, said enzyme having the sequence SEQ ID NO: 4, or functional fragment thereof, or of a functional mutated form thereof,
   ii) an enzyme that is a recombinant protein consisting of the catalytic domain of the endoglucanase EG1 of T. reesei, said enzyme having the sequence SEQ ID NO: 12, or functional fragment thereof, or of a functional mutated form thereof,
   iii) a signal peptide, placed at the N-terminal end of said fusion protein upstream from the two enzymes mentioned in i) and ii), wherein signal peptide is originated from the native cellobiohydrolase mentioned in i), and said signal peptide having the sequence SEQ ID NO: 2,
   iv) a polysaccharide binding module originating from the native cellobiohydrolase mentioned in i), said polysaccharide binding module having the sequence SEQ ID NO: 8 and
      each constituent i), ii) and iv) is linked to one or two of the other constituents i), ii) and iv) at most, by at least one linker peptide of identical or different sequences made up of 10 to 100 amino acids, wherein said fusion proteins has the sequence SEQ ID NO: 14 or a functional mutated form thereof.

In an advantageous embodiment of the method, the enzymatic cocktail and the fusion protein are secreted directly in the hydrolysis medium by T. reesei.

Examples of cellulosic or lignocellulosic substrates are: agricultural and forest residues, herbaceous plants including graminae, wood, including hard wood, soft wood or resinous wood, vegetable pulps such as tomato or sugar beet pulp, low-value biomass such as solid municipal waste (in particular recycled paper), annual crops and dedicated crops. The bioethanol production method comes within the scope of so-called 2^{nd} generation processes. The cellulosic or lignocellulosic substrates used are obtained from essentially non-food resources.

In an even more advantageous embodiment, the fungi mentioned in b) are selected independently of one another among the group consisting of: Aspergillus fumigatus, Aspergillus niger, Aspergillus tubingensis, Chaetomium globosum, Halocyphina villosa, Magnaporthe grisea, Phanerochaete chrysosporium, Pycnoporus cinnabarinus, Pycnoporus sanguineus, Trichoderma reesei.

In another, still more advantageous embodiment of the invention, the ethanol production method according to any of the above definitions is a method wherein the catalytic domain of the cellulase mentioned in ii) has the sequence SEQ ID NO: 2 encoded by SEQ ID NO: 1, corresponding to the catalytic domain of the Endoglucanase EG1 (EG1^{cat}) of T. reesei.

In another more advantageous embodiment of the invention, the ethanol production method according to any one of the above definitions is a method wherein the enzyme mentioned in i) has the sequence SEQ ID NO: 4, corresponding to the catalytic domain of the exo-cellobiohydrolase CBH1 of T. reesei.

In another, still more advantageous embodiment of the invention, the ethanol production method according to any one of the above definitions is a method wherein the cellulosic or lignocellulosic materials have a dry matter content ranging between 3 and 30%, preferably between 5 and 20%.

Finally, in another embodiment of the invention, even more advantageous, the ethanol production method according to any one of the above definitions is a method wherein the fusion protein used in stage b) has as the complete sequence SEQ ID NO: 14 encoded by SEQ ID NO: 13, or a functional mutated form thereof.

Examples 1 to 3 and FIGS. 1 to 6 illustrate the invention.

FIG. 1 illustrates the structure of the CBH1-EG1^{cat} fusion protein as prepared according to Example 1; cat=catalytic domain ; CBM=polysaccharide binding module (Carbohydrates Binding Module).

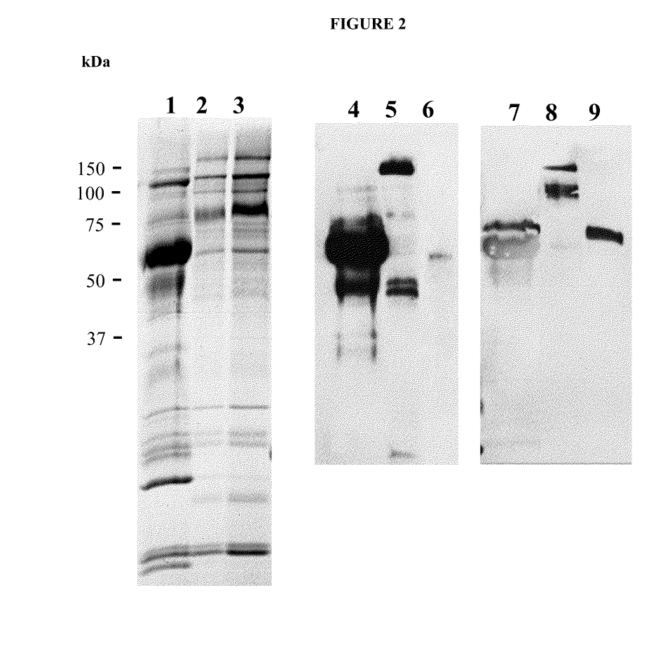

FIG. 2 shows the results of the electrophoresis of the CBH1-EG1^{cat} fusion protein: Coomassie stained gel (columns 1-3) and Western Blot analysis with the anti-EG1 antibodies (columns 4-6) or the anti-CBH1 antibodies (columns 7-9). Columns 1, 4 and 7: CL847Δcbh1 (5 μg); columns 2, 3, 5 and 8: CL847Δcbh1 expressing the CBH1-EG1^{cat} fusion protein, column 6: purified protein EG1 (100 ng), column 9: purified protein CBH1 (200 ng).

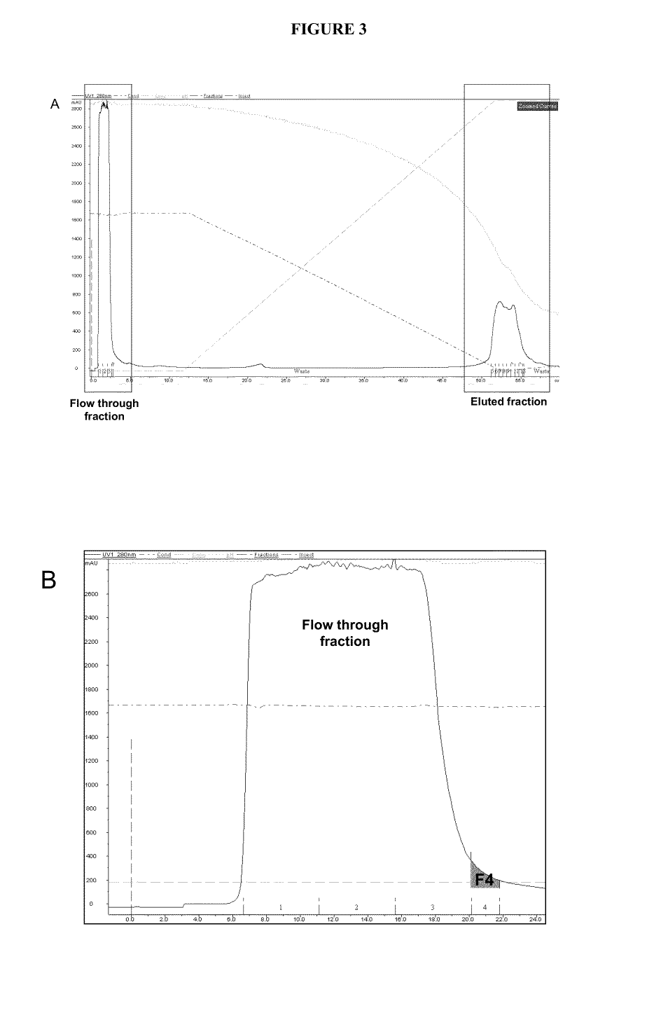

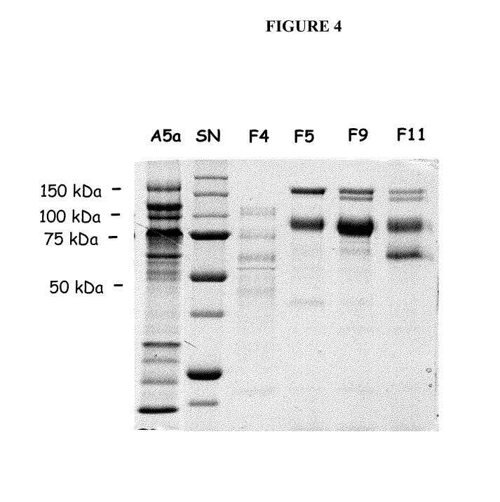

FIG. 3A illustrates the fractionation of the fusion protein according to the technique described in Example 2. FIG. 3B corresponds to the flow-through fraction indicating fraction F4 deposited on gel in FIG. 4.

FIG. 4 represents the SDS-PAGE gel of the supernatant of CL847Δcbh1 expressing the CBH1-EG1^{cat} (A5a SN) fusion protein and of the main fractions collected according to Example 2 (fraction (F) 4, 5, 9 and 11).

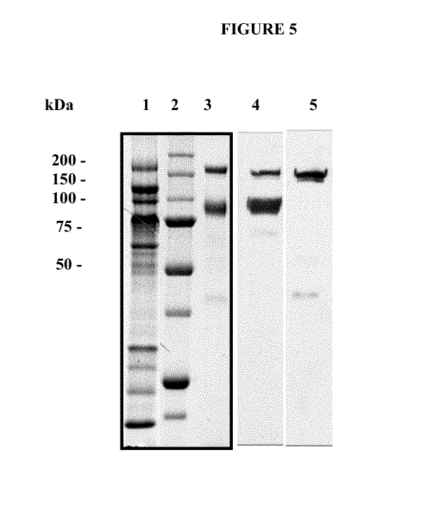

FIG. 5 represents the 10-μSDS-PAGE gel of the culture supernatant (column 1), of the 10-μl molecular marker (column 2) of the CBH1-EG1 purified fusion protein (column 3) and the Western Blot of the purified fusion protein with the anti-CBH1 antibody (column 4) and with the anti-EG1 antibody (column 5).

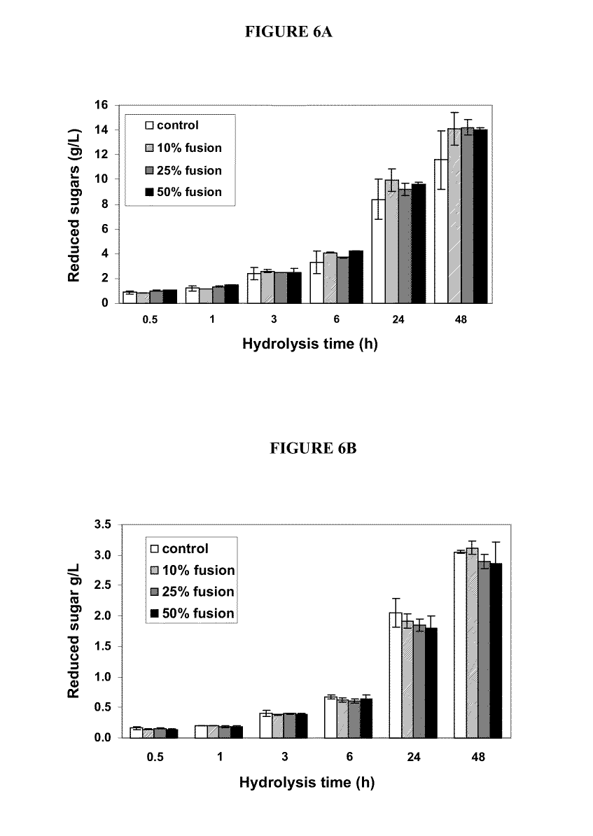

FIGS. 6A and 6B illustrate the hydrolysis yields of wheat straw, steam exploded, by Econase® alone or mixed with increasing amounts of fusion enzyme. FIG. 6A relates to a wheat straw having a dry matter content of 5% and FIG. 6B to a wheat straw having a dry matter content of 1%. The values represent the mean of two samples. CBH1: Cellobiohydrolase 1, EG1: Endoglucanase 1.

### EXAMPLE 1

### Construction of the Fusion Protein and its Expression in T. reesei

The gene coding the CBH1-EG1 fusion protein was cloned in vector pUT1040 under the control of the cbh1 promoter for the expression in strain T. reesei deficient in gene cbh1 (CL847Δcbh1). The CBH1-EG1 fusion protein consists of the entire CBH1 enzyme bound to the coding sequence of the catalytic domain of EG1 by means of the linker peptide of CBH2.

The structure of the fusion protein is illustrated in FIG. 1.

2 clones were obtained (CBH1-EG1_pUT1040) and, after isolation, a clone turned out to be stable (strain A5a). This strain was cultivated on an induction medium (2% lactose/cellulose Solka-Floc® in a Tris-maleate buffer at pH 6) for 3 days. The supernatant was concentrated, washed twice with a citrate buffer and loaded on a SDS-PAGE gel.

The results are given in FIG. 2. A slight band is observed at about 160 kDa in the converted strain that reacts both with the antibodies directed against EG1 and those directed against CBH1, which is absent in the parent strain. The intense band at about 60 kDa in the supernatant of strain CL847Δcbh1 corresponds to the CBH2 that reacts with the anti-EG1 antibody.

### EXAMPLE 2

### Production of the CBH1-EG1^{cat} Fusion Protein Integrated in Strain A5a and Purification by Ion-Exchange Chromatography

Strain A5a is cultivated in a 1.5-L fermenter at 27° C. and at pH 4.8. Biomass production is carried out from a 15 g/l glucose solution as the carbon source. After 30 hours, a continuous flow is started by adding a 250 g/l lactose solution at a flow rate of 2 ml/h. After 215 hours, the protein concentration has reached 9.3 g/l and the supernatant has a filter paper activity of 4.9 FPU/min. The culture is harvested and centrifuged. About 150 ml supernatant are purified by means of a protocol in two stages.

For preliminary purification, the samples are passed through a Hi-Trap® desalting column (5 ml, Biorad) balanced with an acetate buffer. Chromatography is carried out on an AKTA® (GE Healthcare) Mono Q column equilibrated with the same buffer.

The fixed proteins are eluted by a pH gradient by using a PB74 Polybuffer (GE Healthcare) buffer at constant flow rate.

The results are given in FIG. 3.

The grey fractions are analyzed on SDS gel and the results are given in FIG. 4.

The fusion protein is eluted on several fractions, but always simultaneously with smaller proteins. The number and the intensity of these smaller bands increase with the elution process. After concentration, 35 ml purified protein at a concentration of 0.7 mg/ml (including the degradation product) are finally obtained.

In order to determine the identity of the smallest product of 90 kDa that is co-eluted with the fusion protein at 160 kDa, fraction F5 containing the CBH1-EG1^{cat} fusion protein is analyzed by Western blotting. The results are given in FIG. 5, which shows that the two proteins react with the antibody of CBH1, suggesting that the smaller band corresponds to the degradation product. This smaller protein is not recognized by the antibody of EG1 (column 5), indicating that the degradation product has lost its catalytic domain EG1.

### EXAMPLE 3

### Hydrolysis Tests by Increasing Amounts of Fusion Protein CBH1-EG1^{cat}

These tests were carried out with the fusion product obtained in Example 1.

Steam-exploded wheat straw is suspended in a 50-mM citrate buffer at pH 4.8, at a dry matter concentration of 1 or 5%. After adding 32 μl of a 10 g/l tetracycline solution to prevent contamination, the suspensions are brought to equilibrium at 45° C. 12.6 μl Beta-glucosidase (at 25 IU/g dry matter) are added, as well as an enzymatic cocktail of T. reesei (Econase®, from Roal, Finland) with 2.5 mg/g dry matter. In three parallel tests, the Econase is replaced by 10, 25 or 50% (wt. %) fusion enzyme. The samples are stirred at 45° C. and 175 rpm for 2 days and samples are taken at 30 min, 1 h, 3 h, 6 h, 24 h and 48 h. Approximately 500 μl are taken each time and the enzymes are inactivated by boiling for 5 minutes. After centrifugation, the supernatant is filtered through a 0.2-μm filter and stored at −20° C. until analysis. The reduced sugars are measured by means of a DNS test with glucose as the standard.

The results are given in FIGS. 6A and 6B.

After 48 hours, the amount of reduced sugars is increased in the presence of a 10, 25 or 50% (wt. %) mixture of enzymatic cocktail and fusion proteins in comparison with the enzymatic cocktail alone, this result being statistically significant for wheat straw with a dry matter content of 5%.

1. Fusion proteins degrading plant cell walls, said proteins comprising:
i) an enzyme that is a recombinant protein consisting of the catalytic domain of the exo-cellobiohydrolase CBH1, said enzyme having the sequence SEQ ID NO: 4, or functional fragment thereof, or of a functional mutated form thereof,
ii) an enzyme that is a recombinant protein consisting of the catalytic domain of the endoglucanase EG1, said enzyme having the sequence SEQ ID NO: 12, or functional fragment thereof, or of a functional mutated form thereof,
iii) a signal peptide, placed at the N-terminal end of said fusion protein upstream from the two enzymes mentioned in i) and ii), said signal peptide originating from fungal native cellulase or hemicellulase, or from native fungal cellulase belonging to the GH6 or GH7 family,
iv) a polysaccharide binding module originating from fungal native cellulase or hemicellulase, or from native fungal cellulase belonging to the GH6 or GH7 family and
each constituent i), ii) and iv) is linked to one or two of the other constituents i), ii) and iv) at most, by at least one linker peptide of identical or different sequences made up of 10 to 100 amino acids.

2. Fusion proteins degrading plant cell walls according to claim 1, said proteins comprising:
i) an enzyme that is a recombinant protein consisting of the catalytic domain of the exo-cellobiohydrolase CBH1, said enzyme having the sequence SEQ ID NO: 4, or functional fragment thereof, or of a functional mutated form thereof,
ii) an enzyme that is a recombinant protein consisting of the catalytic domain of the endoglucanase EG1, said enzyme having the sequence SEQ ID NO: 12, or functional fragment thereof, or of a functional mutated form thereof,
iii) a signal peptide, placed at the N-terminal end of said fusion protein upstream from the two enzymes mentioned in i) and ii), wherein signal peptide is originated from the native cellobiohydrolase mentioned in i), and said signal peptide having the sequence SEQ ID NO: 2,
iv) a polysaccharide binding module originating from the native cellobiohydrolase mentioned in i), wherein polysaccharide binding module has the sequence SEQ ID NO: 8 and
each constituent i), ii) and iv) is linked to one or two of the other constituents i), ii) and iv) at most, by at least one linker peptide of identical or different sequences made up of 10 to 100 amino acids, wherein said fusion proteins has the sequence SEQ ID NO: 14 or a functional mutated form thereof.

3. A mixture for degrading plant cell walls, comprising a fusion protein as claimed in claim 1. and an enzymatic cocktail of T. reesei.

4. Isolated nucleic acid coding for a fusion protein as claimed in claim 2, said isolated nucleic acids having the sequence SEQ ID NO: 13.

5. An expression vector comprising the nucleic acid molecule as claimed in claim 4 that is functionally linked thereto.

6. A host cell containing the expression vector as claimed in claim 5, said host cell being a cell of a fungus belonging to:
the ascomycetes, including the Aspergillus, Chaetomium, Magnaporthe, Podospora, Neurospora and Trichoderma genera, or
the basidiomycetes, including the Halocyphina, Phanerochaete and Pycnoporus genera.

7. A method of preparing a fusion protein comprising:
i) an enzyme that is a recombinant protein consisting of the catalytic domain of the exo-cellobiohydrolase CBH1, said enzyme having the sequence SEQ ID NO: 4, or functional fragment thereof, or of a functional mutated form thereof,
ii) an enzyme that is a recombinant protein consisting of the catalytic domain of the endoglucanase EG1, said enzyme having the sequence SEQ ID NO: 12, or functional fragment thereof, or of a functional mutated form thereof,
iii) a signal peptide, placed at the N-terminal end of said fusion protein upstream from the two enzymes mentioned in i) and ii), said signal peptide originating from fungal native cellulase or hemicellulase, or from native fungal cellulase belonging to the GH6 or GH7 family,
iv) a polysaccharide binding module originating from fungal native cellulase or hemicellulase, or from native fungal cellulase belonging to the GH6 or GH7 family and
each constituent i), ii) and iv) is linked to one or two of the other constituents i), ii) and iv) at most, by at least one linker peptide of identical or different sequences made up of 10 to 100 amino acids, the method comprising:
in vitro cultivation of the host cell as claimed in claim 6, and
recovery, optionally followed by purification of the fusion protein produced by said host cell.

8. A method of producing ethanol from cellulosic or lignocellulosic materials, comprising:
a) at least one cellulosic or lignocellulosic substrate pretreatment stage,
b) at least one stage of enzymatic hydrolysis of the pretreated substrate, then at least one stage of alcoholic fermentation of the hydrolysate obtained, wherein the enzymatic hydrolysis is carried out by the mixture of an enzymatic cocktail of a fungus secreted by a Trichoderma reesei strain and of a fusion protein consisting of two enzymes degrading the plant cell walls, said fusion protein representing between 1 and 50 wt. %, advantageously between 10 and 50 wt. % of said enzymatic cocktail and comprising:
i) an enzyme that is a recombinant protein consisting of the catalytic domain of the exo-cellobiohydrolase CBH1, said enzyme having the sequence SEQ ID NO: 4, or functional fragment thereof, or of a functional mutated form thereof,
ii) an enzyme that is a recombinant protein consisting of the catalytic domain of the endoglucanase EG1, said enzyme having the sequence SEQ ID NO: 12, or functional fragment thereof, or of a functional mutated form thereof,
iii) a signal peptide, placed at the N-terminal end of said fusion protein upstream from the two enzymes mentioned in i) and ii), said signal peptide originating from fungal native cellulase or hemicellulase, or from native fungal cellulase belonging to the GH6 or GH7 family,
iv) a polysaccharide binding module originating from fungal native cellulase or hemicellulase, or from native fungal cellulase belonging to the GH6 or GH7 family and
each constituent i), ii) and iv) is linked to one or two of the other constituents i), ii) and iv) at most, by at least one linker peptide of identical or different sequences made up of 10 to 100 amino acids.

9. A method of producing ethanol from cellulosic or lignocellulosic materials according to claim 8, comprising:
a) at least one cellulosic or lignocellulosic substrate pretreatment stage,
b) at least one stage of enzymatic hydrolysis of the pretreated substrate, then at least one stage of alcoholic fermentation of the hydrolysate obtained, wherein the enzymatic hydrolysis is carried out by the mixture of an enzymatic cocktail of a fungus secreted by a Trichoderma reesei strain and of a fusion protein consisting of two enzymes degrading the plant cell walls, said fusion protein representing between 1 and 50 wt. %, advantageously between 10 and 50 wt. % of said enzymatic cocktail and comprising:
i) an enzyme that is a recombinant protein consisting of the catalytic domain of the exo-cellobiohydrolase CBH1, said enzyme having the sequence SEQ ID NO: 4, or functional fragment thereof, or of a functional mutated form thereof,
ii) an enzyme that is a recombinant protein consisting of the catalytic domain of the endoglucanase EG1, said enzyme having the sequence SEQ ID NO: 12, or functional fragment thereof, or of a functional mutated form thereof,
iii) a signal peptide, placed at the N-terminal end of said fusion protein upstream from the two enzymes mentioned in i) and ii), wherein signal peptide is originated from the native cellobiohydrolase mentioned in i), and said signal peptide having the sequence SEQ ID NO: 2,
iv) a polysaccharide binding module originating from the native cellobiohydrolase mentioned in i), wherein polysaccharide binding module has the sequence SEQ ID NO: 8 and
each constituent i), ii) and iv) is linked to one or two of the other constituents i), ii) and iv) at most, by at least one linker peptide of identical or different sequences made up of 10 to 100 amino acids, wherein said fusion proteins has the sequence SEQ ID NO: 14 or a functional mutated form thereof.

10. A method as claimed in claim 8, wherein the cellulosic or lignocellulosic materials have a dry matter content ranging between 3 and 30%, preferably between 5 and 20%.

11. A mixture for degrading plant cell walls, comprising a fusion protein as claimed in claim 2, and an enzymatic cocktail of T. reesei.

12. A method as claimed in claim 9, wherein the cellulosic or lignocellulosic materials have a dry matter content ranging between 3 and 30%, preferably between 5 and 20%.


SEQUENCE LISTING (14 sequences)

SEQ ID NO: 1 - DNA, 51 bp, Trichoderma reesei
  CDS (1)..(51): Nucleic acid coding for Trichoderma reesei CBH1 exo-cellobiohydrolase signal peptide
atgtatcggaagttggccgtcatctcggccttcttggccacagctcgtgct

SEQ ID NO: 2 - protein, 17 aa, Trichoderma reesei
MYRKLAVISAFLATARA

SEQ ID NO: 3 - DNA, 1308 bp, Trichoderma reesei
  CDS (1)..(1308): Nucleic acid coding for Trichoderma reesei CBH1 exo-cellobiohydrolase signal peptide
cagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggagttcaccctgcttggcaacgagttctctttcgatgttgatgtttcgcagctgccgtgcggcttgaacggagctctttacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagagttcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaac

SEQ ID NO: 4 - protein, 436 aa, Trichoderma reesei
QSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGN

SEQ ID NO: 5 - DNA, 75 bp, Trichoderma reesei
  CDS (1)..(75): Nucleic acid coding for Trichoderma reesei CBH1 exo-cellobiohydrolase linker peptide
cctcccggcggaaacccgcctggcaccaccaccacccgccgcccagccactaccactggaagctctcccggacct

SEQ ID NO: 6 - protein, 25 aa, Trichoderma reesei
PPGGNPPGTTTTRRPATTTGSSPGP

SEQ ID NO: 7 - DNA, 108 bp, Trichoderma reesei
  CDS (1)..(108): Nucleic acid coding for Trichoderma reesei CBH1 exo-cellobiohydrolase polysaccharide binding module
acccagtctcactacggccagtgcggcggtattggctacagcggccccacggtctgcgccagcggcacaacttgccaggtcctgaacccttactactctcagtgcctg

SEQ ID NO: 8 - protein, 36 aa, Trichoderma reesei
TQSHYGQCGGIGYSGPTVCASGTTCQVLNPYYSQCL

SEQ ID NO: 9 - DNA, 132 bp, Trichoderma reesei
  CDS (1)..(132): Nucleic acid coding for Trichoderma reesei CBH2 exo-cellobiohydrolase linker peptide
cccggcgctgcaagctcaagctcgtccacgcgcgccgcgtcgacgacttctcgcgtatcccccacaacatcccggtcgagctccgcgacgcctccacctggttctactactaccagagtacctccagtcgga

SEQ ID NO: 10 - protein, 44 aa, Trichoderma reesei
PGAASSSSSTRAASTTSRVSPTTSRSSSATPPPGSTTTRVPPVG

SEQ ID NO: 11 - DNA, 1134 bp, Trichoderma reesei
  CDS (1)..(1134): Nucleic acid coding for Trichoderma reesei EG1 Endoglucanase catalytic domain
cagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactccagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcaattgtga

SEQ ID NO: 12 - protein, 377 aa, Trichoderma reesei
QQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAQL

SEQ ID NO: 13 - DNA, 2808 bp, Trichoderma reesei
  CDS (1)..(2808): Nucleic acid coding for full length CBH1-EG1cat fusion protein
atgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggagttcaccctgcttggcaacgagttctctttcgatgttgatgtttcgcagctgccgtgcggcttgaacggagctctttacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagagttcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgcccggcgctgcaagctcaagctcgtccacgcgcgccgcgtcgacgacttctcgcgtatcccccacaacatcccggtcgagctccgcgacgcctccacctggttctactactaccagagtacctccagtcggacagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactccagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcaattgtga

SEQ ID NO: 14 - protein, 935 aa, Trichoderma reesei
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTQSHYGQCGGIGYSGPTVCASGTTCQVLNPYYSQCLPGAASSSSSTRAASTTSRVSPTTSRSSSATPPPGSTTTRVPPVGQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAQL